(19) United States
(12) Patent Application Publication
(10) Pub. No.: US 2025/0248986 A1
(43) Pub. Date: Aug. 7, 2025

(54) ANTIFUNGAL DRUG INHALATION FORMULATIONS

(71) Applicant: Hefei Cosource Pharmaceuticals Co., Ltd, Hefei (CN)

(72) Inventors: Yuting JIN, Hefei (CN); Yihua WANG, Hefei (CN); Dengjun CHEN, Hefei (CN); Xiaoling WANG, Hefei (CN); Xiao WANG, Hefei (CN); Qiongxia XIE, Hefei (CN)

(21) Appl. No.: 19/047,596

(22) Filed: Feb. 6, 2025

(30) Foreign Application Priority Data
Feb. 7, 2024 (CN) 202410176179.X

(51) Int. Cl. A61K 31/454; A61K 9/00; A61K 9/19; A61K 47/12; A61K 47/18

(52) U.S. Cl. CPC A61K 31/454; A61K 9/0075; A61K 9/19; A61K 47/12; A61K 47/183

(57) ABSTRACT

The present disclosure provides an antifungal drug inhalation formulation, comprising: crystalline nanoparticles of a triazole antifungal drug. The present disclosure further provides a preparation method and use of the antifungal drug inhalation formulation.

### CROSS-REFERENCE TO RELATED APPLICATIONS

The present application claims priority to and benefits of Chinese Patent Application No. 202410176179.X filed before the China National Intellectual Property Administration on Feb. 7, 2024, the entire contents of which are incorporated herein by reference.

### TECHNICAL FIELD

The present application relates to the field of pharmaceuticals, and more specifically, to a pharmaceutical inhalation formulation for the treatment of invasive pulmonary fungal infections, allergic bronchopulmonary aspergillosis, and chronic pulmonary aspergillosis, and preparation method thereof.

### BACKGROUND

Among triazole antifungal drugs, itraconazole (ITZ) is a lipophilic triazole broad-spectrum antifungal drug with three chiral centers in the molecular structure and is a racemate mixture consisting of four isomers in a ratio of 1:1:1:1.

Itraconazole can be used for superficial and deep systemic fungal infections, and has good clinical effects and wide application. This drug exerts an antifungal effect through an action mechanism of blocking the ergosterol biosynthetic pathway, which results in increased membrane permeability, decreased activity of membrane-associated enzymes, and external leakage of important substances from the cells, causing death of fungi. Itraconazole has a broad spectrum of antifungal activity against dermatophytes, aspergillus, candida, dematiaceous fungi, histoplasma, coccidioides, blastomycetes, and the like.

Existing itraconazole drugs are primarily administered orally or systemically, but have limited exposure to the lung cavity as well as a variety of adverse reactions and drug-drug interactions. It may be an advantageous option to develop antifungal inhalation formulations with a high concentration in the lung tissues and a low systemic concentration. Currently, there is no commercially available itraconazole inhalation formulation available for inhalation treatment of pulmonary fungal infections.

### SUMMARY OF INVENTION

In a first aspect, the present application provides an antifungal drug inhalation formulation, comprising: crystalline nanoparticles of a triazole antifungal drug.

In some embodiments, the triazole antifungal drug is one of fluconazole, itraconazole, and posaconazole, or the triazole antifungal drug is itraconazole or posaconazole.

In some embodiments, the antifungal drug inhalation formulation further comprises at least one of oleic acid and glycine.

In some embodiments, the average particle size of the crystalline nanoparticles of the triazole antifungal drug ranges from 500 nm to 1,000 nm, further from 100 nm to 800 nm, and even further from 200 nm to 500 nm.

In some embodiments, the antifungal drug inhalation formulation is an inhalation suspension, which further comprises the following ingredients:
a surfactant;
a good solvent and an anti-solvent;
an optional steric stabilizer; and
an optional osmotic pressure regulator.

In some embodiments, the antifungal drug inhalation formulation is an inhalation suspension, which further comprises the following ingredients:
a surfactant;
an acidic pH regulator;
an alkaline pH regulator;
a good solvent and an anti-solvent;
an optional steric stabilizer; and
an optional osmotic pressure regulator.

In some embodiments, the surfactant is one or more selected from the group consisting of macrogol 15 hydroxystearate, glycocholic acid, oleic acid, poloxamer, lecithin, Tween 80, and vitamin E polyethylene glycol succinate.

In some embodiments, the steric stabilizer is one or more selected from the group consisting of hypromellose, polyvinyl alcohol, povidone, copovidone, sodium carboxymethyl cellulose, polyoxyethylene, sodium alginate, and chitosan.

In some embodiments, the osmotic pressure regulator is one or more selected from the group consisting of glycine, mannitol, trehalose, sucrose, lactose, sodium chloride, glucose, cysteine, and lysine.

In some embodiments, the good solvent is one or more selected from the group consisting of propanediol, polyethylene glycol, tert-butanol, and ethanol.

In some embodiments, the anti-solvent is water.

In some embodiments, the acidic pH regulator is one or more selected from the group consisting of hydrochloric acid, phosphoric acid, maleic acid, citric acid, pamoic acid, formic acid, and acetic acid.

In some embodiments, the alkaline pH regulator is one or more selected from the group consisting of trometamol, sodium hydroxide, meglumine, and sodium citrate.

In some embodiments, the inhalation suspension comprises 0.5 g to 5 g, further 1 g to 2.5 g, even further 1 g to 2 g of the triazole antifungal drug, based on 100 ml of a total volume of the inhalation suspension.

In some embodiments, the antifungal drug inhalation formulation is an inhalation powder, which is prepared through lyophilization of the inhalation suspension of any embodiment above.

In some embodiments, the administration route of the antifungal drug inhalation formulation is selected from the group consisting of oral aerosol inhalation or nasal administration.

In a second aspect, the present application provides a preparation method of an antifungal drug inhalation formulation, comprising a step of performing high-pressure homogenization on an initial suspension of a triazole antifungal drug, during which heating to 40° C. to 90° C. and then cooling to room temperature are performed, to obtain a suspension containing crystalline nanoparticles of the triazole antifungal drug.

In some embodiments, the high-pressure homogenization is performed under a pressure of 300 bar to 20,000 bar, or 500 bar to 12,000 bar.

In some embodiments, heating to 50° C. to 70° C. and then cooling to room temperature are performed.

In some embodiments, the initial suspension is stirred for 0 to 12 h, or 0 to 5 h before the high-pressure homogenization is started.

In some embodiments, the preparation method further comprises the following steps:
(1) dissolving the triazole antifungal drug together with a surfactant and an acidic pH regulator in a good solvent to obtain a good solvent solution;
(2) dissolving an alkaline pH regulator, an optional steric stabilizer, an optional osmotic pressure regulator, and an optional surfactant in an anti-solvent to obtain an anti-solvent solution; and
(3) adding the good solvent solution obtained in step (1) to the anti-solvent solution obtained in step (2) to obtain the initial suspension, optionally, adding the good solvent solution obtained in step (1) to the anti-solvent solution obtained in step (2) under stirring.

In some embodiments, at least one of oleic acid and glycine is added in step (1) and/or step (2).

In some embodiments, the triazole antifungal drug is one of fluconazole, itraconazole, and posaconazole, or the triazole antifungal drug is itraconazole or posaconazole.

In some embodiments, the surfactant is one or more selected from the group consisting of macrogol 15 hydroxystearate, glycocholic acid, oleic acid, poloxamer, lecithin, Tween 80, and vitamin E polyethylene glycol succinate.

In some embodiments, the steric stabilizer is one or more selected from the group consisting of hypromellose, polyvinyl alcohol, povidone, copovidone, sodium carboxymethyl cellulose, polyoxyethylene, sodium alginate, and chitosan.

In some embodiments, the osmotic pressure regulator is one or more selected from the group consisting of glycine, mannitol, trehalose, sucrose, lactose, sodium chloride, glucose, cysteine, and lysine.

In some embodiments, the good solvent is one or more selected from the group consisting of propanediol, polyethylene glycol, tert-butanol, and ethanol.

In some embodiments, the anti-solvent is water.

In some embodiments, the acidic pH regulator is one or more selected from the group consisting of hydrochloric acid, phosphoric acid, maleic acid, citric acid, pamoic acid, formic acid, and acetic acid.

In some embodiments, the alkaline pH regulator is one or more selected from the group consisting of trometamol, sodium hydroxide, meglumine, and sodium citrate.

In some embodiments, the preparation method further comprises a step of aseptically filling the suspension containing the crystalline nanoparticles of the triazole antifungal drug and/or lyophilizing the suspension containing the crystalline nanoparticles of the triazole antifungal drug to obtain a lyophilized pharmaceutical formulation.

In a third aspect, the present application provides an antifungal drug inhalation formulation obtained by the preparation method of any embodiment above, wherein optionally, the average particle size of the crystalline nanoparticles of the triazole antifungal drug ranges from 500 nm to 1,000 nm, further from 100 nm to 800 nm, and even further from 200 nm to 500 nm.

In a fourth aspect, the present application provides use of the antifungal drug inhalation formulation of the first aspect or the antifungal drug inhalation formulation of the third aspect in the manufacture of a medicament for the prevention or treatment of invasive pulmonary fungal infections, allergic bronchopulmonary aspergillosis, or chronic pulmonary aspergillosis.

### BRIEF DESCRIPTION OF DRAWINGS

The embodiments illustrated herein are further described below with reference to the accompanying drawings, which, however, are intended merely to enable those skilled in the art to better understand the present application and are not intended to limit the scope of the present application.

FIG. 1 shows the result of microimaging of an itraconazole inhalation liquid formulation prepared according to Comparative Example 1 (direct homogenization of the active pharmaceutical ingredient).

FIG. 2 shows the result of microimaging of an itraconazole inhalation liquid formulation prepared according to Example 1.

FIG. 3 shows the comparison results of powder X-ray diffraction patterns of crystalline forms of the active ingredients in itraconazole inhalation liquid formulations prepared according to Examples 1 to 5 and 9.

FIG. 4 shows a powder X-ray diffraction pattern of a crystalline form of the active ingredient in a posaconazole inhalation liquid formulation prepared according to Example 19.

FIG. 5 shows the electron microscope result of the itraconazole inhalation liquid formulation prepared according to Example 1.

FIG. 6 shows drug plasma concentration-time profiles in the plasma of SD rats for itraconazole inhalation liquid formulations prepared according to Examples 1 to 3 and Comparative Examples 1 and 2, an itraconazole injection for inhalation, and the itraconazole injection for intravenous use.

FIG. 7 shows drug concentration-time profiles in the lung tissue of SD rats for itraconazole inhalation liquid formulations prepared according to Examples 1 to 3 and Comparative Examples 1 and 2, an itraconazole injection for inhalation, and the itraconazole injection for intravenous use.

FIG. 8 shows drug concentration-time profiles in the plasma and the lung tissue of SD rats for the itraconazole inhalation liquid formulation prepared according to Example 1.

FIG. 9 shows drug concentration-time profiles in the plasma and the lung tissue of SD rats for an itraconazole injection after aerosol inhalation.

### DESCRIPTION OF EMBODIMENTS

Hereinafter, the invention concepts of the present application will be further elucidated according to specific embodiments. However, the listed specific embodiments are for illustrative purposes only and are not intended to limit the scope of the present application. Those skilled in the art will recognize that a specific feature in any of the embodiments below may be used in any other embodiments as long as it does not depart from the invention concepts described herein.

Unless otherwise indicated, all numbers indicating sizes, quantities, and physicochemical properties of features used in the specification and claims should be understood to be modified by the term “about” in all instances. Thus, unless stated to the contrary, the numerical parameters set forth in the specification and the claims are approximations, which may be appropriately varied by those skilled in the art under the teachings disclosed herein in order to seek for the desired characteristics. The utilization of a numerical range represented by endpoints includes all numbers within the range and any range within the range, for example, 1 to 5 includes 1, 1.1, 1.3, 1.5, 2, 2.75, 3, 3.80, 4, and 5, etc.

The triazole antifungal drug mainly comprises fluconazole, itraconazole, posaconazole, and the like.

Itraconazole (ITZ) is a lipophilic triazole broad-spectrum antifungal drug having three chiral centers in the molecular structure and is a racemate mixture consisting of four isomers in ratio of 1:1:1:1.

Itraconazole can be used for superficial and deep systemic fungal infections, and has good clinical effects and wide application. This drug exerts an antifungal effect through an action mechanism of blocking the ergosterol biosynthetic pathway, which results in increased membrane permeability, decreased activity of membrane-associated enzymes, and external leakage of important substances from the cells, causing death of fungi. Itraconazole has a broad spectrum of antifungal activity against dermatophytes, aspergillus, candida, dematiaceous fungi, histoplasma, coccidioides, blastomycetes, and the like. Studies have shown that itraconazole has considerable antimicrobial activity against almost all Candida species with a minimum inhibitory concentration (MIC) of ≤1 mg/L against 96% of the Candida species, and strong antimicrobial activity against Aspergillus species with a MIC of ≤1 mg/L against 94% of the Aspergillus species.

However, drug interaction studies have shown that drugs that reduce gastric acid would affect the absorption of itraconazole and that liver drug enzyme inducers can significantly promote the metabolism of itraconazole. Moreover, itraconazole affects the metabolism of other drugs by inhibiting the liver drug enzyme activity, and also has an effect of inhibiting P-glycoprotein, and thereby affecting the absorption of oral drugs, and the high exposure of itraconazole in the systemic circulation of the oral or intravenous route of administration may cause clinical interaction with other basic drugs of the patient, thereby limiting the medication selectivity of the patient. In clinical use, the intravenous rejection of itraconazole requires close attention to the blood concentration of itraconazole.

Itraconazole belongs to the BCS II class of drugs and exhibits the characteristics of low solubility and high permeability. Moreover, itraconazole is a poorly water-soluble weak base with a water solubility of about 1 ng/ml under neutral conditions. The preparation of itraconazole as an inhalation nano suspension can enable the enrichment of the drug in the lung, and prolong the action time of the drug at the lesion site, showing a lower blood concentration than oral or intravenous formulations, and reducing the systematic toxic side effects.

Posaconazole belongs to the second generation of triazole antifungal drugs. Through chemical structural modifications, posaconazole has a side chain extended from the triazole parent ring, chlorine in the benzene ring is substituted by fluorine, and the side chain is hydroxylated, so that posaconazole has a stronger affinity with fungi, higher stability, increased cytotoxicity, and increased water solubility while retaining the lipophilicity of the drug, and it is easier for posaconazole to enter tissues and to be released. In addition, the long side chain structure increases the affinity of the drug to the target (CYP51), so that posaconazole has less effect on the CYP450 enzyme and is only a CYP3A4 inhibitor with higher drug activity and higher safety. Moreover, posaconazole suffers less influence from the 14α-demethylase codon mutation, has a lower binding force to the transmembrane transporter (efflux pump), and is less susceptible to drug resistance, and thus it provides more options for clinical prevention and treatment of invasive fungal diseases (IFD).

In one aspect, the present application provides an antifungal drug inhalation formulation comprising: crystalline nanoparticles of a triazole antifungal drug.

In some embodiments, the antifungal drug inhalation formulation provided by the present application is an inhalation suspension.

The inventors of the present application found in researches that the suspension of amorphous nanoparticles prepared by dissolving a triazole active ingredient in a good solvent and then precipitating the triazole active ingredient by using an anti-solvent is unstable and has a short storage period, while the crystalline nanoparticles obtained through the method of the present application can significantly improve the stability of the nanoparticle suspension.

Herein, unless otherwise indicated, nanoparticles refer to nanoscale particles of the triazole antifungal drug.

In some embodiments, the triazole antifungal drug is one of fluconazole, itraconazole, and posaconazole, or is itraconazole or posaconazole.

In some embodiments, the antifungal drug inhalation formulation of the present application further comprises at least one of oleic acid and glycine. It was found through researches that the storage stability of the crystalline nanoparticles can be further improved by the addition of at least one of oleic acid and glycine, and the resulting suspension has a long-term stability.

In some embodiments, the active ingredient in the antifungal drug inhalation formulation of the present application is itraconazole, and the X-ray powder diffraction (XRPD) pattern of the crystalline nanoparticles of itraconazole has characteristic absorption peaks at 17.5°±0.2° 2θ and 20.5°±0.2° 2θ. The formation of the crystalline form is advantageous for further improving the stability of the formulation.

In some embodiments, the X-ray powder diffraction (XRPD) pattern of the crystalline nanoparticles of itraconazole has characteristic absorption peaks at 17.5°±0.2° 2θ, 20.5°±0.2° 2θ, and 23.6°±0.2° 2θ.

In some embodiments, the active ingredient in the antifungal drug inhalation formulation of the present application is posaconazole, and the X-ray powder diffraction (XRPD) pattern of the crystalline nanoparticles of posaconazole has characteristic absorption peaks at 17.7°±0.2° 2θ and 19.9°±0.2° 2θ.

In some embodiments, the X-ray powder diffraction (XRPD) pattern of the crystalline nanoparticles of posaconazole has characteristic absorption peaks at 9.8°±0.2° 2θ, 17.7°±0.2° 2θ, 19.9°±=0.2° 2θ, and 22.2°±0.2° 2θ.

In some embodiments, the crystalline nanoparticles of the active ingredient in the antifungal drug inhalation formulation of the present application have a narrow particle size distribution to ensure homogeneity and stability of the drug particles. The diversity of the particle size distribution can be assessed by the polydispersity index (PDI). The larger the PDI, the wider the distribution is. The smaller the PDI, the narrower and the more uniform the distribution is. In some embodiments, the crystalline nanoparticles of the active ingredient in the antifungal drug inhalation formulations of the present application have a PDI value of less than 0.3, indicating a narrower particle size distribution. The suspension with a narrow particle size distribution can increase the bioavailability of the drug and help to avoid particle-related toxicity.

In some embodiments, the crystalline nanoparticles in the antifungal drug inhalation formulation of the present application have an average particle size of about 50 nm to 1000 nm. The optimal particle size for macrophage endocytosis is 1 μm to 3 μm, and the particle size of 50 nm to 1000 nm of the crystalline nanoparticles can greatly reduce the possibility of being cleared by the macrophages. Studies have shown that the crystalline nanoparticles can minimize the clearance by the macrophages, thus the clearance of the drug crystalline nanoparticles by the lung macrophages can be minimized after pulmonary inhalation administration due to the nanometric properties of the drug crystalline nanoparticles, resulting in deep deposition in the lung, smaller airway penetration, more uniform drug distribution, a high drug deposition rate, and more accurate drug distribution and efficacy. Unless otherwise indicated, the particle size or average particle size of the crystals herein refers to the Z-average value measured by the dynamic light scattering technique (DLS).

In some embodiments, the crystalline nanoparticles have an average particle size of about 100 nm to 800 nm, further about 200 nm to 500 nm, such as about 300 nm. The drug particles within this particle size range have a relatively faster dissolution rate, which is advantageous to improve the bioavailability.

In some embodiments, the antifungal drug inhalation formulation of the present application is an inhalation suspension, which further comprises the following ingredients: a surfactant; a good solvent and an anti-solvent; an optional steric stabilizer; and an optional osmotic pressure regulator.

In some embodiments, the antifungal inhalation formulation of the present application is an inhalation suspension, which further comprises the following ingredients: a surfactant; an acidic pH regulator; an alkaline pH regulator; a good solvent and an anti-solvent; an optional steric stabilizer; and an optional osmotic pressure regulator.

During the formation of the suspension of the crystalline nanoparticles, the acidic pH regulator in the organic solvent may promote the dissolution of the triazole active ingredient, while the alkaline pH regulator may facilitate the precipitation of the dissolved active ingredient. The particle size stability of the crystalline nanoparticles is important for the suspension and affects the long-term storage stability. The present application increases the stability of the suspension by adding the steric stabilizer and the surfactant. The osmotic pressure regulator may be used to adjust the osmotic pressure of the formulation to close to the physiological osmotic pressure.

In some embodiments, the surfactant is one or more selected from the group consisting of macrogol 15 hydroxystearate, glycocholic acid, oleic acid, poloxamer, lecithin, Tween 80, and vitamin E polyethylene glycol succinate. In some embodiments, the surfactant is at least one selected from the group consisting of macrogol 15 hydroxystearate, poloxamer, glycocholic acid, and vitamin E polyethylene glycol succinate.

The application further increases the stability of the nano suspension by adding the surfactant to generate a repulsion force between the drug particles. In the nano suspension, when the ionic surfactant adsorbs on the surface of the drug, the hydrophobic moiety of the ionic surfactant adsorbs on the surface of the drug particles, and the hydrophilic moiety of the ionic surfactant forms double electric layers and forms a charge barrier around the drug. When two particles attract each other and the distance therebetween is shortened to a certain value, two electric layers repel each other, preventing aggregation of the particles.

In some embodiments, the steric stabilizer is one or more selected from the group consisting of hypromellose, polyvinyl alcohol, povidone, copovidone, sodium carboxymethyl cellulose, polyoxyethylene, sodium alginate, and chitosan. In some embodiments, the steric stabilizer is selected from the group consisting of hypromellose, polyvinyl alcohol, and copovidone. In some embodiments, the steric stabilizer is hypromellose E5.

The present application increases the steric stability of the crystalline particles by adding the macromolecular polymer as the steric stabilizer. These steric stabilizers maintain the stability of the nano suspension by steric hindrance, primarily by adsorbing on or covering the surface of the drug particles by virtue of the affinity of the hydrophobic group to the drug, while the hydrophilic chain interacts with the dispersion medium and extends outwardly to form a dynamic surface, limiting the movement of the drug particles, thereby maintaining the distance between the drug particles. Further, these macromolecular polymers may increase the viscosity of the dispersion medium, hinder the flow and Brownian motion of the particles, and reduce the collision and aggregation of the particles.

In some embodiments, the good solvent is one or more selected from the group consisting of propanediol, polyethylene glycol, tert-butanol, and ethanol. The good solvents used in the present application are all commonly used biocompatible materials, no additional removal process is required, and good drug safety is provided. It is easy for the active ingredient to be dissolved in the good solvent.

In some embodiments, the anti-solvent is water. The triazole antifungal drugs such as itraconazole and posaconazole have extremely low solubility in water, and thus water may be used as the anti-solvent.

The solubilities of itraconazole and posaconazole in the anti-solvent (e.g., water) and in the good solvent (e.g., propanediol and the like) increases with the increase of the temperature. The active ingredient may be first dissolved in the good solvent at a first temperature, and then the active ingredient may be precipitated out from the solvent by adding the good solvent in which the active ingredient is dissolved to the anti-solvent and lowering the temperature, to obtain a nano suspension (an initial suspension).

Itraconazole is a poorly soluble weak base with a calculated log P value of about 6.2. The water solubility of itraconazole is about 1 ng/ml under neutral conditions and about 1 μg/ml under a condition of pH 1. To prevent germination of fungal spores, the concentration of itraconazole is generally required to be greater than 0.5 μg/g in the lung tissue or greater than 0.5 μg/ml in the blood. Posaconazole has a lower solubility and the free base thereof has a solubility of less than 1 μg/ml in an environment at a pH of about 6.4 or higher.

In some embodiments, the use of the organic solvent in combination with the pH regulator has been found surprisingly to greatly increase the solubility of itraconazole and posaconazole, and provide pharmaceutical formulations with high drug loading.

In some embodiments, the pH after formulation with the addition of the good solvent should not be greater than 2.0, and the pH after formulation with the addition of the anti-solvent should not be smaller than 10.0.

In some embodiments, the acidic pH regulator is one or more selected from the group consisting of hydrochloric acid, phosphoric acid, maleic acid, citric acid, pamoic acid, formic acid, and acetic acid. In some embodiments, the acidic pH regulator is selected from the group consisting of hydrochloric acid and phosphoric acid. In some embodiments, the acidic pH regulator is hydrochloric acid.

In some embodiments, the alkaline pH regulator is one or more selected from the group consisting of trometamol, sodium hydroxide, meglumine, and sodium citrate. In some embodiments, the alkaline pH regulator is selected from the group consisting of trometamol and meglumine. In some embodiments, the alkaline pH regulator is at least one selected from the group consisting of trometamol and sodium hydroxide.

In some embodiments, the osmotic pressure regulator is one or more selected from the group consisting of glycine, mannitol, trehalose, sucrose, lactose, sodium chloride, glucose, cysteine, and lysine. In some embodiments, the osmotic pressure regulator is selected from the group consisting of glycine, mannitol, and lactose. In some embodiments, the osmotic pressure modifier is acetylcysteine.

In some embodiments, the antifungal drug inhalation formulation of the present application is an inhalation suspension having a pH of 4.0 to 8.5. In some embodiments, the pH of the inhalation suspension of the present application is 6.0 to 8.0.

In some embodiments, the inhalation suspension comprises 0.5 g to 5 g, further 1 g to 2.5 g, even further 1 g to 2 g of the triazole antifungal drug, based on a total volume of 100 ml of the inhalation suspension.

In some embodiments, the inhalation suspension comprises 1 g to 20 g, further 2 g to 15 g, even further 7.5 g to 13 g of the good solvent, based on a total volume of 100 ml of the inhalation suspension.

In some embodiments, the inhalation suspension comprises 0.2 g to 5 g, further 0.5 g to 2 g of the surfactant, e.g., 0.2 g or 1 g of the surfactant, based on a total volume of 100 ml of the inhalation suspension.

In some embodiments, the inhalation suspension comprises 0 g to 2 g, further 0.1 g to 2 g, even further 0.2 g to 1 g, even further 0.25 g to 0.7 g of the steric stabilizer, based on a total volume of 100 ml of the inhalation suspension.

In some embodiments, the inhalation suspension comprises 0 g to 5 g, further 0.5 g to 2 g of the osmotic pressure regulator, e.g., 2 g of the osmotic pressure regulator, based on a total volume of 100 ml of the inhalation suspension.

In some embodiments, the inhalation suspension comprises 0.05 g to 1.5 g, further 0.1 g to 1 g, even further 0.35 g to 0.8 g of the acidic pH regulator, based on a total volume of 100 ml of the inhalation suspension.

In some embodiments, the inhalation suspension comprises 0.1 g to 2 g, 0.1 g to 1 g, 0.15 g to 0.35 g, or 0.35 g to 0.8 g of the alkaline pH regulator, based on a total volume of 100 ml of the inhalation suspension.

In some embodiments, the inhalation suspension comprises 0 g to 0.5 g, further 0 g to 0.3 g, even further 0 g to 0.1 g, even further 0 g to 0.075 g, even further 0.05 g to 0.075 g of oleic acid, based on a total volume of 100 ml of the inhalation suspension.

In some embodiments, the inhalation suspension comprises 0 g to 4 g, further 0 g to 2 g, even further 1 g to 2 g of glycine, based on a total volume of 100 ml of the inhalation suspension.

In some embodiments, the antifungal drug inhalation formulation of the present application further comprises pamoic acid. It was found in researches that pamoic acid can be used as the acidic pH regulator, and besides, the addition of pamoic acid (e.g., into the aqueous phase) can increase the particle size stability of the crystalline nanoparticles. For example, the amount of pamoic acid added is 0 to 0.2 g, e.g., 0.2 g, based on a total volume of 100 ml of the inhalation suspension.

In some embodiments, the present application provides an antifungal drug inhalation suspension comprising, based on a total volume of 100 ml of the antifungal drug inhalation suspension, the following ingredients:
a triazole antifungal drug, 0.5 g to 5 g;
a surfactant, 0.2 g to 5 g;
a steric stabilizer, 0 g to 2 g;
an osmotic pressure modifier, 0 g to 5 g;
an acidic pH regulator, 0.05 g to 1.5 g;
an alkaline pH regulator, 0.1 g to 2 g;
oleic acid, 0 g to 0.5 g;
glycine, 0 g to 4 g;
pamoic acid, 0 to 0.2 g;
a good solvent, 1 g to 20 g; and
an anti-solvent as the balance (to finalize 100 ml),
wherein the triazole antifungal drug is in the form of crystalline nanoparticles.

In some embodiments, the antifungal drug inhalation suspension provided by the present application comprises, based on a total volume of 100 ml of the antifungal drug inhalation suspension, the following ingredients:
a triazole antifungal drug, 1 g to 2 g;
a surfactant, 0.5 g to 2 g;
a steric stabilizer, 0.2 g to 1 g;
an osmotic pressure regulator, 0.5 g to 2 g;
an acidic pH regulator, 0.1 g to 1 g;
an alkaline pH regulator, 0.1 g to 1 g.
oleic acid, 0 g to 0.3 g;
glycine, 0 g to 2 g;
pamoic acid, 0 to 0.2 g;
a good solvent, 2 g to 15 g; and
an anti-solvent as the balance (to finalize 100 ml),
wherein the triazole antifungal drug is in the form of crystalline nanoparticles.

In some embodiments, the inhalation suspension comprises, based on a total volume of 100 ml of the inhalation suspension, the following ingredients by weight:
itraconazole, 1 g to 5 g;
a surfactant, 0.2 g to 1 g;
a steric stabilizer, 0.25 g to 0.7 g;
an osmotic pressure regulator, 0 g to 2 g, for example 2 g;
an acidic pH regulator, 0.05 g to 0.2 g;
an alkaline pH regulator, 0.35 g to 0.8 g; and
oleic acid, 0 g to 0.075 g, for example 0.05 g to 0.075 g;
glycine, 0 g to 2 g, for example 1 g to 2 g;
a good solvent, 7.5 g to 13 g; and
an anti-solvent as the balance (to finalize 100 ml),
wherein the itraconazole is in the form of crystalline nanoparticles.

In some embodiments, the inhalation suspension comprises, based on a total volume of 100 ml of the inhalation suspension, the following ingredients by weight:
posaconazole, 2.5 g;
a surfactant, 0.25 g to 1 g;
a steric stabilizer, 0.25 g;
an acidic pH regulator, 0.13 g;
an alkaline pH regulator, 0.15 g to 0.35 g;
oleic acid, 0 g to 0.1 g;
glycine, 0 g to 1 g;
pamoic acid, 0 to 0.2 g;
a good solvent, 13 g; and
an anti-solvent as the balance (to finalize 100 ml),
wherein the posaconazole is in the form of crystalline nanoparticles.

In some embodiments, the antifungal drug inhalation formulation provided by the present application may be an inhalation powder. The inhalation powder can be directly used for inhalation administration after being reformulated with water.

In some embodiments, the inhalation powder provided by the present application may be obtained by lyophilization of the antifungal drug inhalation suspension provided in the present application. After the lyophilization, both the good solvent and the anti-solvent in the suspension may be removed. After the lyophilization, the inhalation powder can be directly used for inhalation administration after being reformulated with sterile water for injection.

In some embodiments, the solvent for clinical compatibility of the inhalation powder is sterile water for injection. A suspension can be obtained again after compatibility with the sterile water for injection. After clinical compatibility, the pH value is between 4.0 and 8.5, for example between 6.0 and 8.0.

In some embodiments, the antifungal drug inhalation formulation provided by the present application is administered by a route selected from the group consisting of oral aerosol inhalation and nasal administration. For example, the route of administration of the antifungal drug inhalation formulation provided by the present application is oral aerosol inhalation.

In some embodiments, when administrated through oral aerosol inhalation, the administration volume is not greater than 8 ml for a dose of 20 mg to 400 mg of the active pharmaceutical ingredient; or the administration volume is not greater than 2 ml for a dose of 20 mg to 40 mg of the active pharmaceutical ingredient.

In some embodiments, when administrated through oral aerosol inhalation, 20 mg to 200 mg of the active ingredient may be inhaled for an inhalation volume of 1 ml to 4 ml, thereby effectively inhibiting fungi, avoiding drug failure caused by drug resistance of fungi, and improving patient compliance.

In some embodiments, the mass concentration of the active ingredient in the antifungal drug inhalation suspension provided by the present application is 2 mg/ml to 50 mg/ml, e.g., 10 mg/ml to 40 mg/ml, or 10 mg/ml to 20 mg/ml. Therefore, the single-dose package volume of the drug solution can be reduced to 2 ml. The reduced volume increases the level of aseptic assurance in the manufacturing process while reducing the scale and production costs, facilitating the production, transportation and storage, and further ensuring the quality thereof, and can also reduce the medication time and improves patient compliance in clinical use.

In some embodiments, the antifungal drug inhalation suspension provided by the present application can be administered via inhalation after being atomized by an atomization device. In some embodiments, the atomization device is selected from the group consisting of a pressurized air atomizer, a vibrating mesh atomizer, and an ultrasonic atomization device. The pressurized air atomizer controls and minimizes the amount of drug wasted by inducing respiration and activating the atomization. The vibrating mesh atomizer has an advantage of a higher inhalable fraction compared to the pressurized air atomizer. The piezoelectric element in the ultrasonic atomization device is in contact with the reservoir, and generates vapor mist by high frequency vibration. The inhalation suspensions provided by the present application can be atomized by inhalation devices of these three principles and can achieve high concentrations in the lung tissues.

In another aspect, the present application provides a preparation method of the antifungal drug inhalation formulation as described above, comprising a step of performing high-pressure homogenization on an initial suspension of a triazole antifungal drug, during which heating to 40° C. to 90° C. and then cooling to room temperature are performed, to obtain a suspension containing crystalline nanoparticles of the triazole antifungal drug. In the preparation method of the present application, “initial suspension” refers to a suspension of amorphous particles (e.g., amorphous nanoparticles) of the triazole antifungal drug.

In some embodiments, the high-pressure homogenization is performed under a pressure of 300 bar to 20,000 bar, or 500 bar to 12,000 bar, for example 500 bar, 1,200 bar, or 12,000 bar.

In some embodiments, heating to 50° C. to 70° C. and then cooling to room temperature are performed.

In some embodiments, the initial suspension is stirred for 0 to 12 hours or for 0 to 5 hours before the high-pressure homogenization is started.

In some embodiments, the preparation method of the present application further comprises the steps of:
(1) dissolving the triazole antifungal drug together with a surfactant and an acidic pH regulator in a good solvent to obtain a good solvent solution;
(2) dissolving an alkaline pH regulator, an optional steric stabilizer, an optional osmotic pressure regulator, and an optional surfactant in an anti-solvent to obtain an anti-solvent solution; and
(3) adding the good solvent solution obtained in step (1) to the anti-solvent solution obtained in step (2) to obtain the above-mentioned initial suspension.

In some embodiments, the good solvent solution obtained in step (1) is added to the anti-solvent solution obtained in step (2) under stirring.

In some embodiments, at least one of oleic acid and glycine is added in step (1) and/or step (2).

In some embodiments, pamoic acid is added in step (2) to further increase the stability of the particle size of the formulation.

In some embodiments, the dissolution temperature in step (1) is 30° C. to 80° C., for example, 50° C. to 60° C.

In some embodiments, the dissolution temperature in step (2) is 0 to 40° C., e.g., 0 to 10° C., 0 to 5° C., 10° C. to 20° C., 20° C. to 30° C., or 30° C. to 40° C.

In some embodiments, in step (3), after the good solvent solution is added and before the high-pressure homogenization is started, stirring for 0 to 12 h, for example, 0 to 5 h, e.g., 0 h, 1 h, 2 h, 5 h, 8 h, 10 h, or 12 h, is performed. For example, the stirring for 0 h means that the high-pressure homogenization is started immediately after the good solvent solution obtained in step (1) is added into the anti-solvent solution obtained in step (2) under stirring; and the stirring for 1 h means that stirring is continued for 1 h after the good solvent solution obtained in step (1) is added into the anti-solvent solution obtained in step (2) under stirring, and then the high-pressure homogenization is started.

In some embodiments, the preparation method of the present application further comprises: aseptically filling the suspension containing the crystalline nanoparticles and/or lyophilizing the suspension containing the crystalline nanoparticles to obtain a lyophilized pharmaceutical formulation.

In another aspect, the present application provides an antifungal drug inhalation formulation prepared by the above preparation method. Optionally, the crystalline nanoparticles of the triazole antifungal drug have an average particle size of 50 nm to 1000 nm, further 100 nm to 800 nm, and even further 200 nm to 500 nm, for example, about 300 nm.

In another aspect, the present application provides the use of the antifungal drug inhalation formulation as described above in the manufacture of a medicament for the prevention or treatment of invasive pulmonary fungal infections (IPFI), allergic bronchopulmonary aspergillosis (ABPA), or chronic pulmonary aspergillosis (CPA).

In another aspect, the present application provides a method for treating invasive pulmonary fungal infection (IPFI), allergic bronchopulmonary aspergillosis (ABPA), or chronic pulmonary aspergillosis (CPA), comprising administering to a subject in need thereof (preferably, a mammal, e.g., a human being) the antifungal drug inhalation formulation provided in any of the above embodiments.

The invasive pulmonary fungal infections (IPFI) are classified into two types: the primary type and the secondary type. Common fungi causing IPFI are mainly Candida, Aspergillus, Cryptococcus, zygomycetes (which mainly refers to Mucor), and pneumocystis, etc. Among them, the infectious diseases caused by invasion of Aspergillus mycelium into the lung parenchyma is the invasive pulmonary aspergillosis (IPA).

The allergic bronchopulmonary aspergillosis (ABPA) is an allergic pulmonary disease caused by the sensitization of Aspergillus, most commonly Aspergillus fumigatus. ABPA occurs more often secondary to asthma or pulmonary cystic fibrosis. In addition, ABPA may also occur in patients suffering from other lung diseases, such as bronchiectasis, chronic obstructive pulmonary diseases (COPD), and the like.

The chronic pulmonary aspergillosis (CPA) is a chronic pulmonary infectious disease caused by aspergillus, with a course of more than 3 months. The CPA is classified into 5 types according to the Guide for the Diagnosis and Management of Chronic pulmonary aspergillosis published in Europe in 2015: aspergilloma (SPA), chronic cavitary pulmonary aspergillosis (CCPA), chronic fibrotic pulmonary aspergillosis (CFPA), aspergillosis nodules, and subacute invasive pulmonary aspergillosis. CPA is prevalent in patients with respiratory diseases (e.g., COPD, bronchiectasis, tuberculosis, pulmonary nodules) or mild immunosuppression.

### EXAMPLES

Unless otherwise specified, the drugs or reagents used in the following Examples and Comparative Examples are conventional commercially available products.
Itraconazole: purchased from Sichuan Renan Pharmaceutical Co., Ltd., batch number: 2111003;
Posaconazole: purchased from Shandong Jincheng Kunlun Pharmaceutical Co., Ltd., batch number: 07002303002; and
Hydrochloric acid: in a concentration of 37 wt %.

### Comparative Example 1 (Direct Homogenization)

| Raw materials | Weighed amount | |
|---|---|---|
| Itraconazole | 2 | g |
| Hydrogenated soybean lecithin | 0.2 | g |
| Tween 80 | 0.04 | g |
| Citric acid | 0.585 | g |
| Sodium chloride | 0.6 | g |
| Water | 75 | ml |
| Total volume | 100 | ml |

### Preparation Process:

1. Tween 80, hydrogenated soy lecithin, and sodium chloride were weighed, water was added to achieve dissolution by stirring, and the resulting solution was adjusted to pH 6.5 to 7.5 with citric acid.

2. Itraconazole was weighed, added with the solution of step 1, and stirred to obtain a uniform suspension.

3. The initial suspension obtained in step 2 was homogenized by a high-pressure homogenizer under 1200 Bar, to obtain a fine suspension, which was then filled.

### Comparative Example 2

| | Design quantity- |
|---|---|
| Raw materials | 50 vials |

| | | | |
|---|---|---|---|
| Organic | Itraconazole | 1 | g |
| phase | Kolliphor HS15 | 2 | g |
| | Cholesterol | 0.2 | g |
| | Oleic acid | 0.1 | g |
| | Hydrochloric acid | 0.2 | ml |
| | N,N-dimethylacetamide | 2 | ml |
| Aqueous | Hypromellose E5 | 0.25 | g |
| phase | Trometamol | 0.4 | g |
| | Glycine | 2 | g |
| | Ethylenediamine tetraacetic acid | 0.1 | g |
| | Water (ml) | 80 | ml |

| | | |
|---|---|---|
| Total volume (ml) | 100 | ml |

### Preparation Process:

1. Itraconazole, cholesterol, oleic acid, and macrogol 15 hydroxystearate (Kolliphor HS15) were weighed, N, N-dimethylacetamide and hydrochloric acid were added thereto, and the resulting mixture was stirred in a 60° C. water bath to achieve dissolution, to obtain an organic phase for later use.

2. Hypromellose E5, trometamol, glycine, and ethylenediamine tetraacetic acid were weighed, added into 80 ml of water, stirred to achieve dissolution, and cooled to 0 to 10° C. in a cold water bath for later use.

3. Mixing of two phases and homogenization: the organic phase was aspirated and injected into the aqueous phase (maintained in the cold water bath) under stirring to obtain a white suspension, which was immediately homogenized by a high-pressure homogenizer with circulation under a pressure of 1200 Bar. During the homogenization, the feed liquid was warmed to 40° C., cooling water was then introduced to cool the liquid to room temperature, and water was added to finalize 100 ml.

### Comparative Example 3 (Example 1 of Patent Publication CN101961313B)

| Raw materials | | Design amount | |
|---|---|---|---|
| Acidic | Itraconazole | 2 | g |
| phase | 3 mol/L Hydrochloric acid | 60 | ml |
| | Anhydrous ethanol | 40 | ml |
| Alkaline | Hypromellose 50cp | 0.2 | g |
| phase | Sodium hydroxide | 6 | g |
| | Water | 200 | ml |

| | | |
|---|---|---|
| Total volume | 300 | ml |

### Preparation Process:

1. Itraconazole was weighed, anhydrous ethanol and 3 mol/L hydrochloric acid were added thereto, and the mixture was stirred in a 60° C. water bath to achieve dissolution, to obtain an acidic phase for later use.

2. Hypromellose (50 cp) and sodium hydroxide were weighed, added into 200 ml of water, and stirred to achieve dissolution, to obtain an alkaline phase for later use.

3. Mixing of two phases and homogenization: the acidic phase was aspirated, injected into the alkaline phase under stirring at a speed of 50 r/min, and stirred uniformly to obtain a nano suspension.

### Comparative Example 4 (Example 2 of Patent Publication CN101961313B)

| Raw materials | | Design amount | |
|---|---|---|---|
| Acidic | Itraconazole | 2 | g |
| phase | Hypromellose 50 cp | 1 | g |
| | 3 mol/L Hydrochloric acid | 100 | ml |
| | Water | 80 | ml |
| Alkaline | Hypromellose 50 cp | 1 | g |
| phase | Sodium hydroxide | 6 | g |
| | Water | 200 | ml |

| | | |
|---|---|---|
| Total volume | 380 | ml |

### Preparation Process:

1. Itraconazole and hypromellose were weighed, 3 mol/L hydrochloric acid and water were added thereto, and the resulting mixture was stirred in a 60° C. water bath to achieve dissolution, to obtain an acidic phase for later use.

2. Hypromellose (50 cp) and sodium hydroxide were weighed, added into 200 ml of water, and stirred to achieve dissolution, to obtain an alkaline phase for use.

3. Mixing of two phases: the acidic phase was aspirated and injected into the alkaline phase under stirring at a speed of 500 r/min to obtain a nano suspension.

### Comparative Example 5 (Example 10 of Patent Publication CN101961313B)

| Raw materials | | Design Amount | |
|---|---|---|---|
| Acidic | Itraconazole | 2 | g |
| phase | 3 mol/L Hydrochloric acid | 20 | ml |
| | Anhydrous ethanol | 20 | ml |
| Alkaline | Povidone K90 | 2 | g |
| phase | Poloxamer P407 | 2 | g |
| | Mannitol | 2 | g |
| | Potassium hydroxide | 4 | g |
| | Water | 200 | ml |

| | | |
|---|---|---|
| Total volume | 240 | ml |

### Preparation Process:

1. Itraconazole was weighed, and anhydrous ethanol and hydrochloric acid were added to dissolve the itraconazole under stirring in a 60° C. water bath to obtain an acidic phase for later use.

2. Povidone K90, poloxamer P407, mannitol, and potassium hydroxide were weighed, added into 200 ml of water, and stirred to achieve dissolution, to obtain an alkaline phase for later use.

3. Mixing of two phases: the acidic phase was aspirated and injected into the alkaline phase (maintained in a cold water bath) under stirring at a speed of 200 r/min to obtain a nano suspension.

### Example 1

| | | Design quantity- | |
|---|---|---|---|
| Raw materials | | 50 vials | |
| Organic | Itraconazole | 2 | g |
| phase | Kolliphor HS15 | 1 | g |
| | Hydrochloric acid | 0.4 | ml |
| | Oleic acid | 0.05 | g |
| | Propanediol | 7.5 | ml |
| Aqueous | Hypromellose E5 | 0.25 | g |
| phase | Trometamol | 0.8 | g |
| | Glycine | 2 | g |
| | Water | 80 | ml |

| | | |
|---|---|---|
| Total volume | 100 | ml |

### Preparation Process:

1. Itraconazole and macrogol 15 hydroxystearate (Kolliphor HS15) were weighed, and oleic acid, propanediol, and hydrochloric acid were added thereto and stirred in a 60° C. water bath to achieve dissolution to obtain an organic phase for later use.

2. Hypromellose E5, trometamol, and glycine were weighed, added into 80 ml of water, stirred to achieve dissolution, and cooled to 0 to 5° C. in an ice water bath to obtain an aqueous phase for later use.

3. Mixing of two phases and homogenization: the organic phase was aspirated and injected into the aqueous phase (maintained in the ice water bath) under stirring to obtain a white suspension, which was immediately homogenized by a high-pressure homogenizer for 5 cycles under a pressure of 1200 Bar. During the homogenization, the feed liquid was warmed to 50° C., cooling water was then introduced to cool the liquid to room temperature, and water was added to finalize 100 ml.

4. The obtained 100 ml of suspension was filled in 5 ml vials with 2 ml/vial, plugged, and capped.

### Example 2

| | Design quantity- |
|---|---|
| Raw materials | 150 vials |

| | | | |
|---|---|---|---|
| Organic | Itraconazole | 6 | g |
| phase | Kolliphor HS15 | 2.4 | g |
| | Hydrochloric acid | 0.6 | ml |
| | Tert-butanol | 60 | ml |
| Aqueous | Sodium carboxymethyl cellulose | 1.5 | g |
| phase | Trometamol | 1.2 | g |
| | Glycine | 5.25 | g |
| | Polyethylene glycol 4000 | 3 | g |
| | Water | 210 | ml |

| | | |
|---|---|---|
| Total volume | 300 | ml |

### Preparation Process:

1. Itraconazole and Kolliphor HS15 were weighed, tert-butanol and hydrochloric acid were added thereto, and the mixture was stirred in a 60° C. water bath to achieve dissolution, to obtain an organic phase for later use.

2. Sodium carboxymethyl cellulose, trometamol, polyethylene glycol 4000, and glycine were weighed, added into 320 ml of water, stirred to achieve dissolution, and cooled to 0 to 5° C. in an ice water bath to obtain an aqueous phase for later use.

3. Mixing of two phases and homogenization: the organic phase was aspirated and injected into the aqueous phase (maintained in the ice water bath) under stirring to obtain a white suspension, which was immediately homogenized by a high-pressure homogenizer with circulation under a pressure of 1200 Bar. During the homogenization, the feed liquid was warmed to 50° C. to 60° C., cooling water was then introduced to cool the liquid to room temperature, and water was added to finalize 300 ml to obtain a suspension.

4. The obtained 300 ml of suspension was filled in 5 ml vials with 2 ml/vial, half plugged, lyophilized in a lyophilizer, plugged, and capped. The lyophilized product was re-suspended with water for injection and the particle size was then determined by dynamic light scattering.

### Example 3

| | | Design quantity- | |
|---|---|---|---|
| Raw materials | | 150 vials | |
| Organic | Itraconazole | 3 | g |
| phase | Poloxamer P407 | 0.6 | g |
| | Hydrochloric acid | 0.6 | ml |
| | Ethanol | 30 | ml |
| Aqueous | Trometamol | 1.2 | g |
| phase | Acetylcysteine | 6 | g |
| | Water | 210 | ml |

| | | |
|---|---|---|
| Total volume | 300 | ml |

### Preparation Process:

1. Itraconazole and poloxamer P407 were weighed, ethanol and hydrochloric acid were added thereto, and the mixture was stirred in a 50° C. water bath to achieve dissolution, to obtain an organic phase for later use.

2. Trometamol and acetylcysteine were weighed, added into 210 ml of water, stirred to achieve dissolution, and cooled to 0 to 5° C. in an ice water bath to obtain an aqueous phase for later use.

3. Mixing of two phases and homogenization: the organic phase was aspirated and injected into the aqueous phase (maintained in the ice water bath) under stirring to obtain a white suspension, which was immediately homogenized by a high-pressure homogenizer for 3 circles under a pressure of 1200 Bar. During the homogenization, the feed liquid was warmed to 50° C., cooling water was then introduced to cool the liquid to room temperature, and water was added to finalize 300 ml.

4. The obtained 300 ml of suspension was filled in 5 ml vials with 2 ml/vial, plugged, and capped.

### Example 4

| | | Design quantity- | |
|---|---|---|---|
| Raw materials | | 50 vials | |
| Organic | Itraconazole | 5 | g |
| phase | Kolliphor HS15 | 2 | g |
| | Hydrochloric acid | 0.4 | ml |
| | Tert-butanol | 10 | ml |
| Aqueous | Polyvinyl alcohol 05-88 | 0.2 | g |
| phase | Povidone | 0.5 | g |
| | Trometamol | 0.4 | g |
| | Glycine | 2 | g |
| | Oleic acid | 0.05 | g |
| | Water | 80 | ml |

| | | |
|---|---|---|
| Total volume | 100 | ml |

### Preparation Process:

1. Itraconazole and Kolliphor HS15 were weighed, tert-butanol and hydrochloric acid were added thereto, and the mixture was stirred in a 60° C. water bath to achieve dissolution, to obtain an organic phase for later use.

2. Povidone, polyvinyl alcohol 05-88, trometamol, glycine, and oleic acid were weighed, added into 80 ml of water, stirred to achieve dissolution, and cooled to 0 to 5° C. in an ice water bath to obtain an aqueous phase for later use.

3. Mixing of two phases and homogenization: the organic phase was aspirated and injected into the aqueous phase (maintained in the ice water bath) under stirring to obtain a white suspension, which was immediately homogenized by a high-pressure homogenizer for 6 circles under a pressure of 1200 Bar. During the homogenization, the feed liquid was warmed to 70° C., cooling water was then introduced to cool the liquid to room temperature, and water was added to finalize 100 ml.

4. The obtained 100 ml of suspension was filled in 5 ml vials with 2 ml/vial, half-plugged, lyophilized in a lyophilizer, plugged, and capped.

### Examples 5 to 8: Investigation of Effects of Duration of Mixing of Two Phases on Drug Particle Size

| | | Design quantity- | |
|---|---|---|---|
| Raw materials | | 200 vials | |
| Organic | Itraconazole | 8 | g |
| phase | Poloxamer | 8 | g |
| | Oleic acid | 0.2 | g |
| | Hydrochloric acid | 1.6 | ml |
| | Propanediol | 30 | ml |
| Aqueous | Hypromellose E5 | 1 | g |
| phase | Trometamol | 2 | g |
| | Glycine | 8 | g |
| | Water | 320 | ml |

| | | |
|---|---|---|
| Total volume | 400 | ml |

### Preparation Process:

1. Itraconazole, oleic acid, and poloxamer were weighed, propanediol and hydrochloric acid were added thereto, and the mixture was stirred in a 50° C. water bath to achieve dissolution, to obtain an organic phase for later use.

2. Hypromellose E5, trometamol, and glycine were weighed, added into 80 ml of water, stirred to achieve dissolution, and cooled to 0 to 10° C. in a cold water bath to obtain an aqueous phase for later use.

3. Mixing of two phases and homogenization: the organic phase was aspirated and injected into the aqueous phase (maintained in the cold water bath) under stirring to obtain a white suspension, which was equally divided into three parts. The three parts were maintained under stirring (in 0 to 10° C. water bath) further for 0 h (Example 5), 1 h (Example 6), 2 h (Example 7), and 5 h (Example 8), respectively, and then homogenized by a high-pressure homogenizer with circulation under a pressure of 1200 Bar. During the homogenization, the feed liquid was warmed to 50° C. or above, cooling water was then introduced to cool the liquid to room temperature, and water was added to finalize 400 ml.

4. The obtained 400 ml of suspension was filled in 5 ml vials with 2 ml/vial, plugged, and capped.

### Examples 9 to 12: Investigation of Effects of Temperature Control During Mixing of Two Phases on Drug Particle Size

| | | Design quantity- | |
|---|---|---|---|
| Raw materials | | 200 vials | |
| Organic | Itraconazole | 8 | g |
| phase | Glycocholic acid | 4 | g |
| | Oleic acid | 0.3 | g |
| | Hydrochloric acid | 1.6 | ml |
| | Propanediol | 30 | ml |
| Aqueous | Copovidone | 1 | g |
| phase | Trometamol | 2.4 | g |
| | Glycine | 4 | g |
| | Water | 320 | ml |

| | | |
|---|---|---|
| Total volume | 400 | ml |

### Preparation Process:

1. Itraconazole, oleic acid, and glycocholic acid were weighed, propanediol and hydrochloric acid were added thereto, and the mixture was stirred in a 60° C. water bath to achieve dissolution, to obtain an organic phase, which was equally divided into four parts for later use.

2. Copovidone, trometamol, and glycine were weighed, added into 320 ml of water, and stirred to achieve dissolution. The resulting mixture was evenly divided into four parts. The four parts were stored in water baths of 0 to 10° C. (Example 9), 10° C. to 20° C. (Example 10), 20° C. to 30° C. (Example 11), and 30° C. to 40° C. (Example 12), respectively, to obtain aqueous phases for later use.

Mixing of two phases and homogenization: the organic phase was aspirated and respectively injected into the aqueous phases at different temperatures (maintained in the water bath) under stirring to obtain white suspensions, which were stirred for 10 min and then homogenized by a high-pressure homogenizer with circulation under a pressure of 1200 Bar. During the homogenization, the feed liquid was warmed to 70° C. or above, cooling water was then introduced to cool the liquid to room temperature, and water was added to finalize 400 ml.

4. The obtained 400 ml of suspension was filled in 5 ml vials with 2 ml/vial, plugged, and capped.

### Examples 13 to 15: Investigation of Effects of Different Steric Stabilizers on Drug Particle Size

| | | Example | | Example | | Example | |
|---|---|---|---|---|---|---|---|
| Raw materials | | 13 | | 14 | | 15 | |
| Organic | Itraconazole | 2 | g | 2 | g | 2 | g |
| phase | Vitamin E polyethylene | 2 | g | 2 | g | 2 | g |
| | glycol succinate | | | | | | |
| | Hydrochloric acid | 0.4 | ml | 0.4 | ml | 0.4 | ml |
| | Propanediol | 10 | ml | 10 | ml | 10 | ml |
| Aqueous | Povidone K30 | 0.5 | g | — | | — | |
| phase | Copovidone VA64 | — | | 0.5 | | — | |
| | Hypromellose E5 | — | | — | | 0.5 | |
| | Trometamol | 0.4 | g | 0.4 | g | 0.4 | g |
| | Glycine | 2 | g | 2 | g | 2 | g |
| | Oleic acid | 0.05 | g | 0.05 | g | 0.05 | g |
| | Water | 80 | ml | 80 | ml | 80 | ml |

| | | | | | | |
|---|---|---|---|---|---|---|
| Total volume | 100 | ml | 100 | ml | 100 | ml |

### Preparation Process:

1. Itraconazole and vitamin E polyethylene glycol succinate were weighed, propanediol and hydrochloric acid were added thereto, and the mixture was stirred in a 60° C. water bath to achieve dissolution to obtain an organic phase for later use.

2. Povidone K30 (or copovidone VA64, or hypromellose E5), trometamol, oleic acid, and glycine were weighed, added into 80 ml of water, stirred to achieve dissolution, and cooled to 0 to 10° C. in a cold water bath to obtain an aqueous phase for later use.

3. Mixing of two phases and homogenization: the organic phase was aspirated and injected into the aqueous phase (maintained in the cold water bath) under stirring to obtain a white suspension, which was immediately homogenized with circulation under a pressure of 1200 Bar. During the homogenization, the feed liquid was warmed to 50° C. or above, cooling water was then introduced to cool the liquid to room temperature, and water was added to finalize 100 ml.

### Examples 16 to 18: Investigation of Effects of Different Proportions of Glycine on Drug Particle Size

| | | Example | | Example | | Example | |
|---|---|---|---|---|---|---|---|
| Raw materials | | 16 | | 17 | | 18 | |
| Organic | Itraconazole | 2 | g | 2 | g | 2 | g |
| phase | Vitamin E polyethylene | 1 | g | 1 | g | 1 | g |
| | glycol succinate | | | | | | |
| | Oleic acid | 0.075 | g | 0.075 | g | 0.075 | g |
| | Hydrochloric acid | 0.4 | ml | 0.4 | ml | 0.4 | ml |
| | Propanediol | 10 | ml | 10 | ml | 10 | ml |
| Aqueous | Hypromellose E5 | 0.25 | g | 0.25 | g | 0.25 | g |
| phase | Trometamol | 0.5 | g | 0.4 | g | 0.4 | g |
| | Glycine | 0 | g | 1 | g | 2 | g |
| | Water | 80 | ml | 80 | ml | 80 | ml |

| | | | | | | |
|---|---|---|---|---|---|---|
| Total volume | 100 | ml | 100 | ml | 100 | ml |

### Preparation Process:

1. Itraconazole, vitamin E polyethylene glycol succinate, and oleic acid were weighed, propanediol and hydrochloric acid were added thereto, and the mixture was stirred in a 60° C. water bath to achieve dissolution to obtain an organic phase for later use.

2. Hypromellose E5, trometamol, and glycine were weighed, added into 80 ml of water, stirred to achieve dissolution, and cooled to 0 to 10° C. in a cold water bath to obtain an aqueous phase for later use.

3. Mixing of two phases and homogenization: the organic phase was aspirated and injected into the aqueous phase (maintained in the cold water bath) under stirring to obtain a white suspension, which was immediately homogenized with circulation under a pressure of 1200 Bar. During the homogenization, the feed liquid was warmed to 50° C. or above, cooling water was then introduced to cool the liquid to room temperature, and water was added to finalize 100 ml.

### Example 19

| | Raw materials | Design amount | |
|---|---|---|---|
| Organic | Posaconazole | 5 | g |
| phase | Oleic acid | 0.2 | g |
| | Poloxamer P188 | 0.5 | g |
| | Hydrochloric acid | 0.6 | ml |
| | Propanediol | 25 | ml |
| Aqueous | Hypromellose E5 | 0.5 | g |
| phase | Trometamol | 0.7 | g |
| | Glycine | 2 | g |
| | Water | 125 | ml |
| | Total volume | 200 | ml |

### Preparation Process:

1. Posaconazole, oleic acid, and poloxamer P188 were weighed, propanediol and hydrochloric acid were added thereto, and the mixture was stirred in a 60° C. water bath to achieve dissolution to obtain an organic phase for later use.

2. Hypromellose E5, trometamol, and glycine were weighed, added into 125 ml of water, stirred to achieve dissolution, and cooled to 0 to 10° C. in a cold water bath to obtain an aqueous phase for later use.

3. Mixing of two phases and homogenization: the organic phase was aspirated and injected into the aqueous phase (maintained in the cold water bath) under stirring to obtain a white suspension, which was immediately homogenized with circulation under a pressure of 1200 Bar. During the homogenization, the feed liquid was warmed to 60° C. or above, cooling water was then introduced to cool the liquid to room temperature, and water was added to finalize 200 ml.

### Example 20

| | Raw materials | Design amount | |
|---|---|---|---|
| Organic | Posaconazole | 5 | g |
| phase | Vitamin E polyethylene glycol succinate | 2 | g |
| | Hydrochloric acid | 0.6 | ml |
| | Propanediol | 25 | ml |
| Aqueous | Hypromellose E5 | 0.5 | g |
| phase | Sodium hydroxide | 0.3 | g |
| | Pamoic acid | 0.2 | g |
| | Water (ml) | 125 | ml |
| | Total volume (ml) | 200 | ml |

### Preparation Process:

1. Posaconazole, oleic acid, and vitamin E polyethylene glycol succinate were weighed, propanediol and hydrochloric acid were added thereto, and the mixture was stirred in a 60° C. water bath to achieve dissolution to obtain an organic phase for later use.

2. Hypromellose E5, sodium hydroxide, and pamoic acid were weighed, added into 125 ml of water, stirred to achieve dissolution, and cooled to 0 to 10° C. in a cold water bath to obtain an aqueous phase for later use.

3. Mixing of two phases and homogenization: the organic phase was aspirated and injected into the aqueous phase (maintained in the cold water bath) under stirring to obtain a white suspension, which was immediately homogenized with circulation under a pressure of 1200 Bar. During the homogenization, the feed liquid was warmed to 60° C. or above, cooling water was then introduced to cool the liquid to room temperature, and water was added to finalize 200 ml.

### Performance Test

Unless otherwise specified, the content of the active ingredient in the formulation was detected under the following high performance liquid chromatography conditions:

The content was determined by high performance liquid chromatography (General Rule 0512) as specified in Part IV of the 2020 edition of the Chinese Pharmacopoeia using octadecylsilane chemically bonded silica as the filler; an acetonitrile-0.02 mol/L tetrabutylammonium hydrogen sulfate solution (40:60) as the mobile phase at a flow rate of 1.5 ml/min; and a detection wavelength of 225 nm. The theoretical plate number is not less than 3000 calculated based on the itraconazole peak, and the resolution between the itraconazole peak and the adjacent impurity peak should meet the requirements. An appropriate amount of an itraconazole reference sample was taken, accurately weighed, added with an appropriate amount of 70% acetonitrile, dissolved ultrasonically, and diluted quantitatively into a solution containing 0.5 mg/ml itraconazole as a reference stock solution, which was diluted with 70% acetonitrile to linear concentrations of 5 μg/ml, 20 μg/ml, 60 μg/ml, 100 μg/ml, 160 μg/ml, and 200 μg/ml as reference solutions. The solutions were injected to the liquid chromatography, and the chromatograms were recorded. The contents were calculated based on the peak area according to the external standard method.

The contents of the active ingredient in the plasma and the tissues were determined by LC-MS in the pharmacokinetic analysis.
Chromatographic conditions of pharmacokinetic assay
Detection system: HPLC-UV (Shimadzu SPD-10AVP, LC-20AD)
Liquid chromatography column: ACE Excel 3 C18 50×3.0 mm
Column temperature: 40° C.
Cleaning solution: Isopropanol:Methanol:Water=1:1:1
Flow rate: 0.5 mL/min
Autosampler temperature: 4° C.
Injection volume: 10 μL
Mobile phase A: 0.1% formic acid in water containing 2 mM ammonium acetate
Mobile phase B: Methanol

### Gradient Elution:

| Time (min) | % B | Flow rate (mL/min) |
|---|---|---|
| 0.00 | 80 | 0.5 |
| 2.00 | 100.0 | 0.5 |
| 2.50 | 100.0 | 0.5 |
| 2.60 | 80 | 0.5 |
| 3.50 | 80 | 0.5 |

### Mass Spectral Conditions:

Mass spectrometry system: LC-MS/MS
Ionization mode: (−) ESI
Scan mode: MRM
Parent ion, fragment ion and collision energy (CE)

| | Precursor | Product | TL | Q1 pre | CE | Q3 pre |
|---|---|---|---|---|---|---|
| TA | 705.200 | 392.300 | 101 | 0.70 | 34 | 0.70 |
| IS | 531.200 | 243.800 | 115 | 0.70 | 31 | 0.70 |

The particle size was measured by the dynamic light scattering technique (DLS) and expressed as a value of the Z-average size.

The polydispersity index (PDI) was measured by a conventional PDI detection instrument.

### Tests of Example 1, Example 3, and Comparative Example 2

The stability of the particle size (Z-average size) and polydispersity index (PDI) of Example 1, Example 3 and Comparative Example 2 was measured.

Test method: The inhalation formulations prepared in Example 1, Example 3 and Comparative Example 2 were each diluted 50 times with water and then placed in the sample cell, and the Z-average size and PDI thereof were measured.

| | Placement | | | Z-average | |
|---|---|---|---|---|---|
| Sample name | condition | Time | | size (d · nm) | PDI |
| Example 1 | — | 0 | | 247.24 | 0.127 |
| | 25° C. | 3 | months | 214.73 | 0.175 |
| | | 6 | months | 232.70 | 0.188 |
| | | 9 | months | 234.24 | 0.172 |
| | | 12 | months | 244.25 | 0.136 |
| | | 24 | months | 219.52 | 0.189 |
| | 40° C. | 3 | months | 254.58 | 0.105 |
| | | 12 | months | 245.20 | 0.175 |
| | | 24 | months | 230.16 | 0.222 |

| | Placement | | | Z-average | |
|---|---|---|---|---|---|
| Sample name | condition | Time | | size (d · nm) | PDI |
| Example 3 | — | 0 | | 360.00 | 0.156 |
| | 25° C. | 3 | months | 353.00 | 0.180 |
| | | 6 | months | 324.54 | 0.144 |
| | | 9 | months | 351.79 | 0.170 |
| | | 12 | months | 338.55 | 0.087 |
| | 40° C. | 3 | months | 381.00 | 0.206 |
| | | 6 | months | 455.00 | 0.158 |
| | | 9 | months | 349.74 | 0.112 |
| | | 12 | months | 560.54 | 0.148 |

| | Placement | | Z-average | |
|---|---|---|---|---|
| Sample name | condition | Time | size (d · nm) | PDI |
| Comparative | — | 0 | 157.77 | 0.233 |
| Example 2 | 25° C. | 3 days | 213.12 | 0.137 |
| | | 7 days | 305.24 | 0.227 |

By comparing Example 1 with Comparative Example 2, it can be seen that Example 1 of the present application is stable in storage at 25° C. with little change in the Z-average size, while Comparative Example 2 used cholesterol and N, N-dimethylacetamide as the good solvents and used the ethylenediamine tetraacetic acid chelating agent, resulting in a suspension with a smaller crystalline particle size (157.77 nm) and unstable storage at 25° C. In addition, experiments showed that itraconazole nanoparticles produced with other bioincompatible solvents, such as dimethylformamide, dimethylsulfoxide, and N-methylpyrrolidone as the good solvent also have smaller Z-average sizes (about 100 nm) and poor stability. By comparing Example 1 with Example 3, it can be seen that the stability of Example 1 with the addition of glycine and oleic acid is superior to that of Example 3 without the addition of glycine and oleic acid.

### Tests of Comparative Examples 3 to 5

The stability of the particle size (Z-average size) and polydispersity index (PDI) of Comparative Examples 3 to 5 were measured.

Test method: The inhalation formulations prepared in Comparative Examples 3 to 5 were each diluted with water and then placed in the sample cell, and the Z-average size and PDI thereof were measured.

| | Placement | | | Z-average | |
|---|---|---|---|---|---|
| Sample name | condition | Time | | size (d · nm) | PDI |
| Comparative | — | 0 | h | 330.21 | 0.189 |
| Example 3 | 25° C. | 2 | h | 482.81 | 0.418 |
| | | 4 | h | 815.52 | 0.451 |
| | | 8 | h | 1288.10 | 0.502 |
| | | 24 | h | 2331.1 | 0.583 |
| Comparative | — | 0 | h | 288.13 | 0.214 |
| Example 4 | 25° C. | 2 | h | 374.43 | 0.306 |
| | | 4 | h | 599.87 | 0.358 |
| | | 8 | h | 828.57 | 0.407 |
| | | 24 | h | 1754.31 | 0.512 |
| Comparative | — | 0 | h | 285.12 | 0.200 |
| Example 5 | 25° C. | 2 | h | 500.10 | 0.188 |
| | | 4 | h | 898.35 | 0.287 |
| | | 8 | h | 988.14 | 0.449 |
| | | 24 | h | 1937.77 | 0.471 |

Test conclusion: the nano solutions prepared according to the examples of Patent CN101961313B have poor particle size stability.

### Tests of Example 1 and Comparative Example 1

The microscope images of Example 1 and Comparative Example 1 were determined.

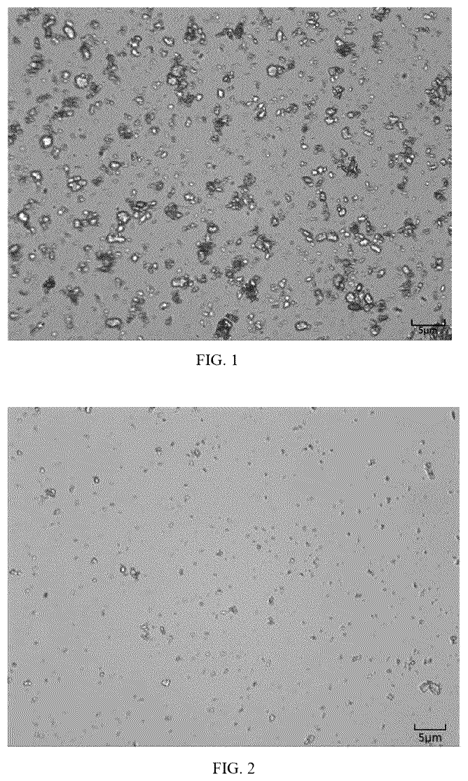

Test method: The inhalation formulations prepared in Comparative Example 1 and Example 1 were each diluted 50 times with water, one drop of the diluted formulation was placed on a glass slide, covered by a cover glass, and then placed on the stage, and the microscope image (400 ×) was collected. The results are shown in FIG. 1 (Comparative Example 1) and FIG. 2 (Example 1), respectively.

In the preparation process of Comparative Example 1, the direct homogenization of the active pharmaceutical ingredient (top-down method) was used, and the particle size of the suspension obtained by the high-pressure homogenization was about 2 μm.

The preparation method of the nanoparticle suspension adopted in the present application ensures that a suspension with a lower particle size is obtained by combining the bottom-up precipitation process and the top-down high-pressure homogenization process. The particle size of Example 1 of the present application was smaller than 250 nm, significantly smaller than that of Comparative Example 1.

### Tests of Examples 1, 2 to 5, 9 and 19

The crystalline forms of Example 1 (0 month and 12 months), Examples 2 to 5, and Example 9 were determined.

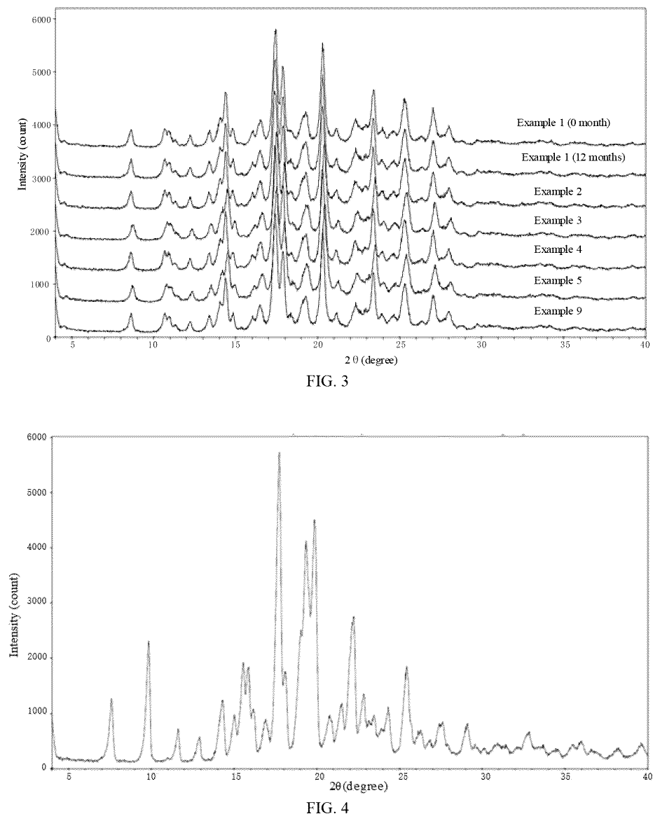

Test method: the inhalation formulations prepared in Example 1 (0 month and 12 months), Examples 2 to 5, and Example 9 were each centrifuged, and then the resulting precipitate was taken and dried at a low temperature to obtain a test sample. The position and relative intensity of the diffraction peaks of the test samples were measured in the range of 4° to 40° by a powder X-ray diffractometer (DX-27 mini mode), and the results are shown in FIG. 3.

The posaconazole liquid inhalation formulation prepared in Example 19 was centrifuged, and then the resulting precipitate was taken and dried at low temperature to obtain a test sample. The position and relative intensity of the diffraction peaks of the test sample were measured in the range of 4° to 40° by a powder X-ray diffractometer (DX-27 mini mode), and the results are shown in FIG. 4.

It can be confirmed that the crystalline form of itraconazole and the crystalline form of posaconazole are obtained, and the crystalline form of itraconazole obtained is stable and can be stored for a long time.

### Tests of Examples 1 to 3 and Comparative Examples 1 and 2

The particle size (Z-average size) and polydispersity index (PDI) of each of Examples 1 to 3 and Comparative Examples 1 and 2 were measured.

Test method: The inhalation formulations prepared in Examples 1 to 3 and Comparative Examples 1 and 2 were each diluted 50 times with water and then placed in the sample cell, and the Z-average size and PDI thereof (standing time: 0 day) were measured.

| | Sample name | | | | |
|---|---|---|---|---|---|
| | Exam- | Exam- | Exam- | Comparative | Comparative |
| | ple 1 | ple 2 | ple 3 | Example 1 | Example 2 |
| Z-average | 247.24 | 573.55 | 360.00 | 2306.54 | 157.77 |
| size (d · nm) | | | | | |
| PDI | 0.127 | 0.186 | 0.156 | — | 0.233 |

It indicates that the particle sizes obtained under different process conditions are different.

### Test of Example 20

The particle size (Z-average size) and polydispersity index (PDI) of Example 20 were determined.

Test method: The inhalation formulation prepared in Example 20 was diluted 50 times with water and then placed in the sample cell, and the Z-average size and PDI thereof were determined.

| Sample name | Example 20 |
|---|---|
| Z-average size (d · nm) | 342.21 |
| PDI | 0.168 |

It indicates that a suspension of nanoparticles of posaconazole was obtained.

### Test of Examples 1 and 2

The aerodynamic particle size distribution of the itraconazole suspension for aerosol inhalation after being atomized by an atomizer was determined with reference to the method in the General Rule 0951 of Part IV of the 2020 Edition of the Chinese Pharmacopoeia. The gas flow rate was set to 15 L/min and the atomization lasted for 2 min. The drug deposited on each part of the instrument (atomizer, throat pipe, collecting cup at each stage) was washed with 70% acetonitrile, placed in a corresponding volumetric flask, finalized to the volume, and shaken evenly. The content of itraconazole was measured, and the amount of drug deposited on each part was calculated. The mass median aerodynamic diameter (MMAD), geometric standard deviation (GSD), FPD (fine particle dose, i.e., the cumulative deposition dose with aerodynamic particle size of less than 5 μm), and FPF<5 μm (fine particle fraction, i.e., the percentage of the cumulative deposition dose with aerodynamic particle size of smaller than 5 μm relative to the output dose) were calculated by the analysis software. The determination results of the aerodynamic particle size are shown in the following table. The drug recovery rates measured were all greater than 95%.

| | Total amount | FPD | FPF | MMAD | |
|---|---|---|---|---|---|
| Bath No. | collected (mg) | (mg) | (%) | (μm) | GSD |
| Example 1 | 4.308 | 2.96 | 67.206 | 3.197 | 2.503 |
| Example 2 | 4.609 | 22.588 | 54.769 | 3.508 | 2.337 |

### Tests of Examples 5 to 8

Test method: The inhalation formulations prepared in Examples 5 to 8 were each diluted 50 times with water and then placed in the sample cell, and the Z-average size and PDI thereof were measured.

| | Example 5 | Example 6 | Example 7 | Example 8 |
|---|---|---|---|---|
| Z-average | | | | |
| size (d · nm) | | | | |
| 0 day | 297.5 | 317.64 | 357.92 | 335.88 |
| 2 months-25° C. | 286.43 | 336.64 | 366.95 | 372.87 |
| PDI | | | | |
| 0 day | 0.23 | 0.21 | 0.258 | 0.269 |
| 2 months-25° C. | 0.238 | 0.264 | 0.303 | 0.395 |

Examples 5 to 8 were investigated for the effects of the duration of mixing of the two phases prior to the high-pressure homogenization on the particle size of the drug. It can be seen that the longer the duration of mixing, the larger the particle size of the final product, and the duration of mixing should be in the range of 0 to 12 h, further 0 to 5 h.

### Tests of Examples 9 to 12

Test method: The inhalation formulations prepared in Examples 9 to 12 were each diluted 50 times with water and then placed in the sample cell, and the Z-average size and PDI thereof were measured.

| | Example 9 | Example 10 | Example 11 | Example 12 |
|---|---|---|---|---|
| Z-average | | | | |
| size (d · nm) | | | | |
| 0 day | 293.95 | 311.17 | 287.66 | 310.92 |
| 1 month-25° C. | 309.71 | 327.56 | 296.22 | 314.42 |
| 2 months-25° C. | 308.14 | 333.57 | 302.48 | 323.69 |
| PDI | | | | |
| 0 day | 0.194 | 0.218 | 0.194 | 0.272 |
| 1 month-25° C. | 0.216 | 0.179 | 0.171 | 0.286 |
| 2 months-25° C. | 0.121 | 0.085 | 0.125 | 0.139 |

It can be seen that Examples 9 to 12 were investigated for the effects of temperature control during the mixing of the two phases on the particle size of the drug, and that the particle size had small variation when the dissolution temperature is within 0 to 40° C.

### Tests of Examples 13 to 15

Test method: The inhalation formulations prepared in Examples 13 to 15 were each diluted 50 times with water and then placed in the sample cell, and the Z-average particle size and PDI thereof were measured.

| | Placement | Z-average | |
|---|---|---|---|
| Batch No. | condition | size (d · nm) | PDI |
| Example 13 | 0 month | 284.31 | 0.183 |
| (Povidone K30) | 25° C. 1 month | 295.35 | 0.204 |
| | 25° C. 2 months | 305.45 | 0.214 |
| | 25° C. 3 months | 322.46 | 0.199 |
| Example 14 | 0 month | 314.58 | 0.190 |
| (Copovidone VA64) | 25° C. 1 months | 319.44 | 0.158 |
| | 25° C. 2 months | 338.49 | 0.168 |
| | 25° C. 3 months | 357.12 | 0.158 |
| Example 15 | 0 month | 222.8 | 0.185 |
| (Hypromellose E5) | 25° C. 1 months | 234.15 | 0.187 |
| | 25° C. 2 months | 240.17 | 0.155 |
| | 25° C. 3 months | 252.44 | 0.167 |
| | 40° C. 4 days | 254.24 | 0.147 |
| | 40° C. 12 days | 245.24 | 0.161 |
| | 60° C. 12 days | 282.35 | 0.164 |

The crystalline nanoparticles in the itraconazole suspensions of inhalation formulations prepared in Examples 13 to 15 using different steric stabilizers were stably stored at room temperature with little change in particle size within 3 months. In addition, in Example 15, hypromellose E5 was selected as the steric stabilizer in the anti-solvent, and the particle size of the obtained inhalation formulation showed an excellent high-temperature stability.

### Tests of Examples 16 to 18

| | Placement | Z-average | |
|---|---|---|---|
| Sample name | condition | size (d · nm) | PDI |
| Example 16 | 0 day | 304.75 | 0.249 |
| (Glycine 0 g/100 ml) | 40° C. 6 days | 382.03 | 0.191 |
| | 40° C. 2 months | 413.2 | 0.189 |
| | 25° C. 1 month | 384.46 | 0.249 |
| | 25° C. 2 months | 399.61 | 0.196 |
| Example 17 | 0 day | 278.56 | 0.14 |
| (Glycine 1 g/100 ml) | 40° C. 6 days | 282.91 | 0.081 |
| | 40° C. 2 months | 289.41 | 0.215 |
| | 25° C. 1 month | 292.46 | 0.216 |
| | 25° C. 2 months | 281.62 | 0.175 |
| Example 18 | 0 day | 304.05 | 0.195 |
| (Glycine 2 g/100 ml) | 40° C. 6 days | 311.07 | 0.151 |
| | 40° C. 2 months | 322.57 | 0.161 |
| | 25° C. 1 month | 317.27 | 0.141 |
| | 25° C. 2 months | 303.52 | 0.17 |

The test results showed that when 1 g to 2 g of glycine was added to the anti-solvent based on a total volume of 100 ml, the obtained inhalation formulation had a good particle size stability.

### Test of Example 1 (Electron Microscope)

Test instrument: Transmission electron microscope/energy spectrometer (TEM-EDS)

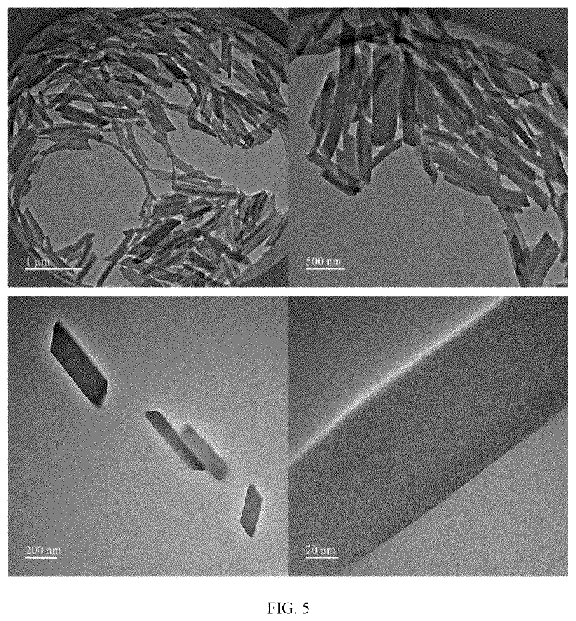

Test method: The suspension prepared in Example 1 was centrifuged (14,000 rpm, 10 min), the supernatant was discarded, the precipitate was re-suspended and washed with a 1.0% aqueous solution of Kolliphor HS15, and centrifuged again (14,000 rpm, 10 min), the supernatant was discarded, and the precipitate was re-suspended with the 1.0% aqueous solution of Kolliphor HS15. The particles in the re-suspended solution were dispersed in a dispersing agent by ultrasonic waves or stirring to form a suspension. The copper mesh covered with a support film was clamped tight by tweezers, a few drops of the suspension was dropped with a dropper onto the surface of the support film, and the clamping continued until the drops were air-dried. Electron micrographs were observed and recorded from the transmission electron microscope/spectrometer. The results are shown in FIG. 5.

It can be seen that the active pharmaceutical ingredient (API) in the inhalation suspension prepared in the present application are prismatic crystals under electron microscopy.

Tests of Examples 1 to 3 and Comparative Examples 1 and 2: Pharmacokinetic Studies in Plasma and Lung Tissues after Aerosol Inhalation in SD Rats

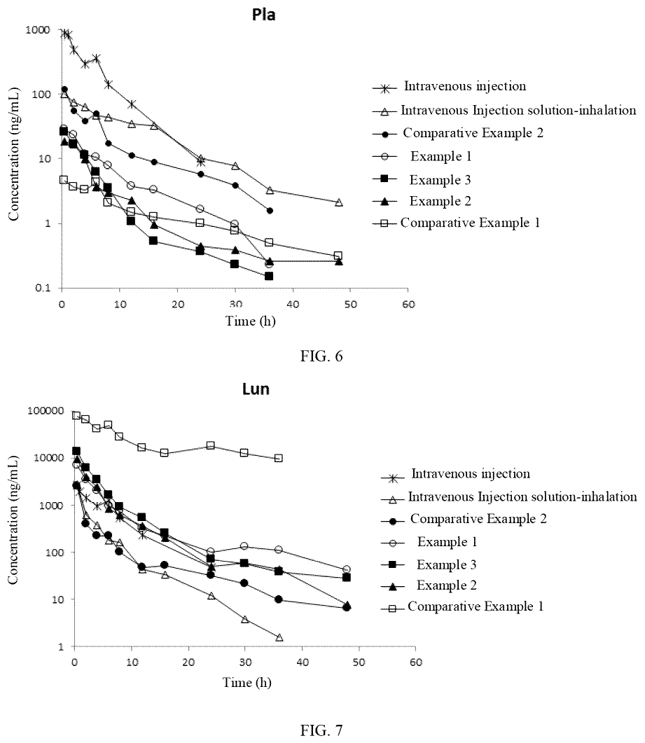

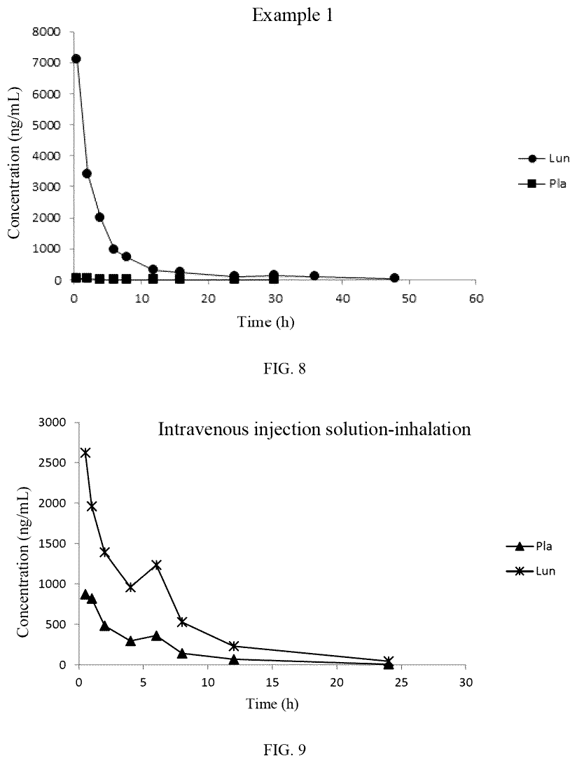

Test method: 136 SD rats (SPF-grade SD rats, the range of body weight: 190 g to 220 g at the time of administration, purchased from Zhejiang Vital River Laboratory Animal Technology Co., Ltd.) were divided into 4 groups, 16 rats in Group 1 for intravenous administration (an itraconazole injection, manufactured by GlaxoSmithKline Manufacturing S.p.A., Packaging Specification 25 ml: 0.25 g×1 bottle/box), and 20 rats in each group of Groups 2 to 7 for inhalation (the itraconazole injection, Examples 1 to 3, and Comparative Examples 1 and 2). The animals were dissected for the collection of the blood and lungs at 0.5 h, 1 h, 2 h, 4 h, 6 h, 8 h, 12 h, and 24 h after administration in the intravenous group, and at 0.5 h, 2 h, 4 h, 6 h, 8 h, 12 h, 24 h, 30 h, 36 h, and 48 h after administration in the inhalation groups. The concentration of itraconazole in the samples was determined by LC-MS/MS and the pharmacokinetic parameters were calculated by WinNonlin 8.2. The drug plasma concentration-time curves in the plasma (Pla) of SD rats (FIG. 6) and the drug concentration-time curves in the lung tissues (Lun) of SD rats (FIG. 7) for the itraconazole inhalation liquid formulations prepared according to Examples 1 to 3 and Comparative Examples 1 and 2, an itraconazole injection for inhalation, and the itraconazole injection for intravenous use, the drug concentration-time curves in the plasma and lung tissues of SD rats for the itraconazole inhalation liquid formulation prepared in Example 1 (FIG. 8), and the drug concentration-time curves in the plasma and lung tissues of SD rats after aerosol inhalation of the itraconazole injection (FIG. 9) were plotted.

The main pharmacokinetic parameters are shown in the following table:

| | | Itraconazole | | | | | | | | | | |
|---|---|---|---|---|---|---|---|---|---|---|---|---|
| | Itraconazole | injection for | Comparative | | Example | | Example | | Example | | Comparative | |
| Category | injection | inhalation | Example 2 | | 1 | | 3 | | 2 | | Example 1 | |
| Z-average size | — | — | 155 | nm | 247.24 | nm | 360.00 | nm | 573.55 | nm | 2 | μm |
| Administration mode | Intravenous | Inhalation | Inhalation | | Inhalation | | Inhalation | | Inhalation | | Inhalation | |
| Dose | 5 mg/kg | 3 mg/kg | 3 | mg/kg | 3 | mg/kg | 3 | mg/kg | 3 | mg/kg | 3 | mg/kg |

| | | | | | | | | |
|---|---|---|---|---|---|---|---|---|
| Blood | AUC(μg/L*h) | 4374.90 | 1107.38 | 550.24 | 185.12 | 113.28 | 107.82 | 50.19 |
| | Tmax(h) | 0.5 | 0.5 | 0.5 | 0.5 | 0.5 | 0.5 | 0.5 |
| | Cmax(μg/L) | 873.54 | 100.80 | 121.08 | 28.24 | 25.85 | 18.71 | 4.54 |
| | t½(h) | 3.991 | 4.205 | 4.757 | 7.842 | 7.981 | 9.723 | 11.712 |
| Lung | AUC(ng/g*h) | 13846.70 | 5855.39 | 4493.04 | 34900.42 | 42549.44 | 56988.03 | 566568.26 |
| | Tmax(h) | 0.5 | 0.5 | 0.5 | 0.5 | 0.5 | 0.5 | 0.5 |
| | Cmax(ng/g) | 2623.22 | 2699.32 | 2531.74 | 7080.43 | 13466.17 | 19185.74 | 75760.21 |
| | t½(h) | 4.695 | 4.212 | 3.593 | 5.644 | 5.941 | 5.946 | 30.11 |

| | | | | | | | |
|---|---|---|---|---|---|---|---|
| AUC | 3.17 | 5.29 | 8.17 | 188.65 | 375.62 | 528.53 | 11289.59 |
| lung-to-blood | | | | | | | |
| ratio | | | | | | | |

Test results: Compared with the formulation for intravenous administration, all of the inhalation formulations can increase the concentration of the drug in the lung tissues, and achieve the purpose of enrichment of the active drug in the lung. At the same time, the exposure of the drug in the plasma is lower for the inhalation formulations, which can avoid the adverse reactions caused by systemic application. The particle size of the inhalation formulation directly affects the pharmacokinetics of the drug. The particle size significantly affects the lung-to-blood ratio of itraconazole in vivo. The inhalation formulation prepared in Comparative Example 1 has a Z-average size of about 2 μm, a high lung-to-blood ratio, but poor permeability, less entry of the drug into the blood, excessively long residence time in the lung tissues, and a half-life of 30.11 h, which might cause local toxicity. The inhalation formulation prepared in Comparative Example 2 has a Z-average size of about 155 nm and is characterized by rapid permeation, a low concentration in the lung, a lung-to-blood ratio of merely 8.17, rapid entry of itraconazole into the blood after inhalation, and a short half-life in the lung, which cannot effectively achieve the goal of high lesion concentration and low systemic exposure. The inhalation of the itraconazole injection also exhibits rapid permeation with a lower concentration in the lung and a lung-to-blood ratio of 5.29. Examples 1 to 3 of the present application have a lung-to-blood ratio in the range of 188.65 to 528.53, which achieves the purpose of enriching the active drug in the lung and reducing the drug exposure in the plasma, and have a half-life of the drug between 5.644 and 5.946 h in the lung tissues, which ensures that the drug can reside in the lung for a period of time to exert its effect without causing toxicity due to long-term residence.

In summary, the preparation of the suspension of crystalline nanoparticles of the present application combines the bottom-up precipitation process and the top-down high-pressure homogenization process to obtain a suspension with a better particle size. The stability of the particle size is important for suspension-type liquid formulations and affects whether the formulations can be stably stored for a long period of time, and the appropriate particle size is the core element for balancing the lung-to-blood ratio, permeability, and the local residence time of the drug. The present application enhances the stability of the nano suspension by using some macromolecular polymers as the steric stabilizer (e.g., povidone, hypromellose E5, etc.). The present application further improves the stability of the nano suspension by adding the surfactant to generate the repulsion force between the drug particles and the like. Further, the selection of the good solvent is critical, the present application uses the good solvent that can be used in the inhalation formulations and has good drug safety, and the concentration of the good solvent in the final product is controlled at a lower level without the need for a removal operation in a subsequent process. By controlling the average particle size in the range of 50 nm to 1000 nm, more preferably 100 nm to 800 nm, and even more preferably 200 nm to 500 nm, the present application is able to achieve the purpose of enriching the drug in the lung tissues to enhance the efficacy without causing toxicity, as well as a lower exposure in the plasma to reduce systemic adverse reactions. Moreover, the suspension of the crystalline nanoparticles in the above particle size range has an excellent storage stability, and in some embodiments, the sample still has good particle size stability after standing at 40° C. for 24 months.

The above are merely specific implementations of inventions encompassed by the present application, and the scope of protection of the present application is not limited thereto. Any variation or substitution easily conceived by those skilled in the art within the scope of techniques disclosed in the present application should be covered within the scope of protection of the present application. Therefore, the scope of protection of this application should be based on the scope of protection of the claims.

1. An antifungal drug inhalation formulation, comprising: crystalline nanoparticles of a triazole antifungal drug.

2. The antifungal drug inhalation formulation according to claim 1, wherein the triazole antifungal drug is one of fluconazole, itraconazole, and posaconazole, or the triazole antifungal drug is itraconazole or posaconazole.

3. The antifungal drug inhalation formulation according to claim 1, further comprising at least one of oleic acid and glycine.

4. The antifungal drug inhalation formulation according to claim 1, wherein an average particle size of the crystalline nanoparticles of the triazole antifungal drug ranges from 500 nm to 1,000 nm, further from 100 nm to 800 nm, and even further from 200 nm to 500 nm.

5. The antifungal drug inhalation formulation according to claim 1, wherein the antifungal drug inhalation formulation is an inhalation suspension, which further comprises the following ingredients:
a surfactant;
a good solvent and an anti-solvent;
an optional steric stabilizer; and
an optional osmotic pressure regulator; or
the antifungal drug inhalation formulation is an inhalation suspension, which further comprises the following ingredients:
a surfactant;
an acidic pH regulator;
an alkaline pH regulator;
a good solvent and an anti-solvent;
an optional steric stabilizer; and
an optional osmotic pressure regulator.

6. The antifungal drug inhalation formulation according to claim 5, wherein
the surfactant is one or more selected from the group consisting of macrogol 15 hydroxystearate, glycocholic acid, oleic acid, poloxamer, lecithin, Tween 80, and vitamin E polyethylene glycol succinate; and/or
the steric stabilizer is one or more selected from the group consisting of hypromellose, polyvinyl alcohol, povidone, copovidone, sodium carboxymethyl cellulose, polyoxyethylene, sodium alginate, and chitosan; and/or
the osmotic pressure regulator is one or more selected from the group consisting of glycine, mannitol, trehalose, sucrose, lactose, sodium chloride, glucose, cysteine, and lysine; and/or
the good solvent is one or more selected from the group consisting of propanediol, polyethylene glycol, tert-butanol, and ethanol; and/or
the anti-solvent is water; and/or
the acidic pH regulator is one or more selected from the group consisting of hydrochloric acid, phosphoric acid, maleic acid, citric acid, pamoic acid, formic acid, and acetic acid; and/or
the alkaline pH regulator is one or more selected from the group consisting of trometamol, sodium hydroxide, meglumine, and sodium citrate.

7. The antifungal drug inhalation formulation according to claim 5, wherein the inhalation suspension comprises 0.5 g to 5 g, further 1 g to 2.5 g, even further 1 g to 2 g of the triazole antifungal drug, based on 100 ml of a total volume of the inhalation suspension.

8. The antifungal drug inhalation formulation according to claim 5, wherein the inhalation suspension is lyophilized into an inhalation powder.

9. The antifungal drug inhalation formulation according to claim 1, wherein an administration route of the antifungal drug inhalation formulation is selected from the group consisting of oral aerosol inhalation and nasal administration.

10. A preparation method of an antifungal drug inhalation formulation, comprising a step of:
performing high-pressure homogenization on an initial suspension of a triazole antifungal drug, during which heating to 40° C. to 90° C. and then cooling to room temperature are performed, to obtain a suspension containing crystalline nanoparticles of the triazole antifungal drug.

11. The preparation method according to claim 10, wherein:
the high-pressure homogenization is performed under a pressure of 300 bar to 20,000 bar, or 500 bar to 12,000 bar; and/or
heating to 50 to 70° C. and then cooling to room temperature are performed;
optionally, the initial suspension is stirred for 0 to 12 h, or 0 to 5 h before the high-pressure homogenization is started.

12. The preparation method according to claim 10, further comprising the following steps:
(1) dissolving the triazole antifungal drug together with a surfactant and an acidic pH regulator in a good solvent to obtain a good solvent solution;
(2) dissolving an alkaline pH regulator, an optional steric stabilizer, an optional osmotic pressure regulator, and an optional surfactant in an anti-solvent to obtain an anti-solvent solution; and
(3) adding the good solvent solution obtained in step (1) to the anti-solvent solution obtained in step (2) to obtain the initial suspension,
optionally, adding the good solvent solution obtained in step (1) to the anti-solvent solution obtained in step (2) under stirring.

13. The preparation method according to claim 12, wherein at least one of oleic acid and glycine is added in step (1) and/or step (2).

14. The preparation method according to claim 10, wherein:
the triazole antifungal drug is one of fluconazole, itraconazole, and posaconazole, or the triazole antifungal drug is itraconazole or posaconazole; and/or
the surfactant is one or more selected from the group consisting of macrogol 15 hydroxystearate, glycocholic acid, oleic acid, poloxamer, lecithin, Tween 80, and vitamin E polyethylene glycol succinate; and/or
the steric stabilizer is one or more selected from the group consisting of hypromellose, polyvinyl alcohol, povidone, copovidone, sodium carboxymethyl cellulose, polyoxyethylene, sodium alginate, and chitosan; and/or
the osmotic pressure regulator is one or more selected from the group consisting of glycine, mannitol, trehalose, sucrose, lactose, sodium chloride, glucose, cysteine, and lysine; and/or
the good solvent is one or more selected from the group consisting of propanediol, polyethylene glycol, tert-butanol, and ethanol; and/or
the anti-solvent is water; and/or
the acidic pH regulator is one or more selected from the group consisting of hydrochloric acid, phosphoric acid, maleic acid, citric acid, pamoic acid, formic acid, and acetic acid; and/or
the alkaline pH regulator is one or more selected from the group consisting of trometamol, sodium hydroxide, meglumine, and sodium citrate.

15. The preparation method according to claim 10, further comprising a step of:
aseptically filling the suspension containing the crystalline nanoparticles and/or lyophilizing the suspension containing the crystalline nanoparticles to obtain a lyophilized pharmaceutical formulation.

16. An antifungal drug inhalation formulation obtained by the preparation method according to claim 10, optionally, an average particle size of the crystalline nanoparticles of the triazole antifungal drug ranging from 500 nm to 1,000 nm, further from 100 nm to 800 nm, and even further from 200 nm to 500 nm.

17. A method for preventing or treating invasive pulmonary fungal infections, allergic bronchopulmonary aspergillosis, or chronic pulmonary aspergillosis, comprising 5 administrating the antifungal drug inhalation formulation according to claim 1 to a subject in need thereof.
